(19) United States
(12) Patent Application Publication
(10) Pub. No.: US 2004/0068766 A1
(43) Pub. Date: Apr. 8, 2004

(54) Enzyme

(76) Inventors: Peter Poulsen, Copenhagen O (DK); Iben Schildt Sorensen, Copenhagen N (DK)

Correspondence Address: Nixon & Vanderhye, 8th Floor, 1100 North Glebe Road, Arlington, VA, 22201-4714, US

(21) Appl. No.: 10/239,145

(22) PCT Filed: Mar. 16, 2001
(86) PCT No.: PCT/IB01/00493

(51) Int. Cl. A01H 1/00; C12N 15/82

(52) U.S. Cl. 800/284

(57) ABSTRACT

The present invention relates to a transformed plant having a reduced endogenous starch branching enzyme (SBE) activity, and having a heterologous glucan branching enzyme (GBE) activity. The invention also relates to starch obtainable from such a plant.

### FIELD OF THE INVENTION

The present invention relates to plants with modified glucan branching enzyme (GBE) activity. The invention also related to starches produced from plants with modified GBE activity.

### BACKGROUND TO THE INVENTION

Starch is one of the main storage carbohydrates in plants, especially higher plants. The structure of starch consists of amylose and amylopectin. Amylopectin comprises linear or branched glucans. Amylose consists essentially of straight chains of α-1-4-linked glycosyl residues. Amylopectin (linear or branched glucans) comprises chains of a-1-4-linked glycosyl residues with some α-1-6 branches. The branched nature of amylopectin is created by the action of, among other things, enzyme(s) known as glucan branching enzyme(s) (“GBE(s)”). GBEs can catalyse the formation of branch points in the amylopectin (linear or branched glucans) molecule, for example by adding α-1,4 glucans through α-1,6-glucosidic branching linkages. GBEs include starch branching enzyme(s) (SBEs), as well as glycogen branching enzyme(s) (GLYBEs). Enzymes such as these may be from any source, for example from prokaryotic and/or eukaryotic sources.

It is known that starch is an important raw material. Starch is widely used in the food, paper, and chemical industries. Moreover, a large fraction of the starches used in these industrial applications are post-harvest modified by chemical, physical or enzymatic methods in order to obtain starches with certain required functional properties. However, such treatments are comparitively costly, and often involve hazardous chemicals. This is a problem associated with the prior art.

Within the past few years it has become desirable to make genetically modified plants which could be capable of producing modified starches which could be the same as the post-harvest modified starches. It is also known that it may be possible to prepare such genetically modified plants by expression of antisense nucleotide coding sequences. In this regard, June Bourque provides a detailed summary of antisense strategies for the genetic manipulations in plants (Bourque 1995 Plant Science 105 pp 125-149). Reviews on how enzymatic activity can be affected by expression of particular nucleotide sequences may be found in the teachings of Finnegan and McElroy [1994] Biotechnology 12 883-888; and Matzke and Matzke [1995] TIG 11 1-3. Whilst it is known that enzymatic activity can be affected by expression of particular nucleotide sequences there is still a need for a method that can more reliably and/or more efficiently and/or more specifically affect enzymatic activity and/or introduce new enzymatic activity into the system.

### SUMMARY OF THE INVENTION

As explained above, it is desirable to produce starches which have certain properties. Starches with these properties are not readily available in plants (in particular commercial crop plants) without post-harvest modification. It is surprisingly shown herein that plants may be genetically modified in order to produce starch with modified properties as compared with starch from the naturally occurring parent plants. The present application discloses that this may be accomplished by the alteration or modification of glucan branching enzyme (GBE) activity in plant tissues.

Accordingly, the present invention provides transformed plants which have modified glucan branching enzyme (GBE) activity. The term ‘transfomed plants’ includes transgenic plants, or plants harbouring transgene construct(s), whether stably or transiently.

GBE activity may be modified by any suitable technique, such as reducing the levels of GBE enzyme(s), such as through the use of an inhibitor of endogenous GBE expression and/or the use of an inhibitor of endogenous GBE replication (eg. transcription/translation); reducing the level of endogenous GBE enzymatic activity, such as through the use of a moiety that alters the active site of the endogenous GBE. Examples of such inhibitors or moieties include enzymes, antibodies, nucleotide sequences, etc. The term ‘modified GBE activity’ thus includes alteration by reduction of endogenous GBE and/or the introduction of heterologous GBE activity and/or the augmentation of endogenous GBE activity such as by modulation, enhancement, reduction or increase of said endogenous GBE activity. Preferably the reduction of endogenous GBE activity refers to the reduction of endogenous SBE activity. Preferably the term ‘endogenous glucan branching enzyme’ (endogenous GBE) refers to ‘endogenous starch branching enzyme’ (endogenous SBE).

In one preferred aspect, the alteration of glucan branching enzyme activity may be accomplished by reduction of endogenous GBE activity using antisense expression.

Preferably, this is accomplished using GBE antisense expression constructs as described hereinbelow, and as shown in the figures. Preferably ‘GBE antisense expression constructs’ refers to ‘SBE antisense expression constructs’.

Antisense expression constructs are nucleic acid constructs which are capable of directing the production of RNA complementary to the coding RNA of a particular gene. Reduction of GBE activity refers to a reduction of the endogenous enzymatic activity, or to a reduction in the levels of endogenous GBE enzyme(s), or to a reduction in the levels of endogenous GBE transcript(s). This reduction of endogenous GBE activity may be accomplished by the use of sense intron expression, antisense intron expression, sense exon expression or antisense exon expression, or any combination thereof. In the case of a combination, the individual expression constructs may be on the same or different nucleic acid molecules, or any combination thereof. Preferably the reduction of endogenous GBE activity may be accomplished by the use of antisense exon expression. Preferably the reduction of endogenous GBE activity refers to the reduction of endogenous SBE activity by use of antisense exon expression, more preferably by use of antisense SBE exon expression.

The present invention also provides that plants having modified GBE activity may be produced by the expression of heterologous GBEs in the tissues of the plant(s). Preferably, plants having modified GBE activity may be produced by both the reduction of endogenous GBE activity using antisense expression, and the expression of heterologous GBEs in the tissues of said plant(s).

Accordingly, the present invention provides transformed plants which have modified glucan branching enzyme (GBE) activity, wherein said activity is brought about by the reduction of endogenous GBE activity, and the heterologous expression of one or more GBE(s).

### DETAILED ASPECTS OF THE PRESENT INVENTION

In one aspect, the present invention relates to a transformed plant having an altered endogenous glucan branching enzyme (GBE) activity, and having a heterologous starch branching enzyme activity.

In another aspect, the present invention relates to a transformed plant having an altered endogenous glucan branching enzyme (GBE) activity, and having a heterologous starch branching enzyme activity, wherein said reduced GBE activity is effected via antisense expression of at least part of an GBE exon.

In another aspect, the present invention relates to a transformed plant having an altered endogenous glucan branching enzyme (GBE) activity, and having a heterologous starch branching enzyme activity, wherein said reduced GBE activity is effected via antisense expression of at least part of one or more SBE I exon(s), or antisense expression of at least part of one or more SBE II exon(s), or antisense expression of of at least part of one or more exon(s) from both SBE I and SBE II.

In another aspect, the present invention relates to a transformed plant having an altered endogenous glucan branching enzyme (GBE) activity, and having a heterologous starch branching enzyme activity, wherein said reduced GBE activity is effected via antisense exon expression, and wherein the antisense exon expression system and the heterologous GBE expression system are present as separate nucleic acid constructs.

In another aspect, the present invention relates to a transformed plant as described herein, wherein said heterologous GBE activity comprises a SBE, or a glycogen branching enzyme (GLYBE), or both an SBE and a GLYBE.

In another aspect, the present invention relates to a transformed plant as described herein, wherein said heterologous GBE activity comprises an GBE obtainable from an alga.

In another aspect, the present invention relates to a transformed plant as described herein, wherein said heterologous GBE activity comprises an GBE obtainable from a red alga.

In another aspect, the present invention relates to a transformed plant as described herein, wherein said heterologous GBE activity comprises an GBE obtainable from one or more algae selected from Gracilaria gracilis and Gracilaria lemaneifonnis.

In another aspect, the present invention relates to a transformed plant as described herein, wherein said heterologous GBE activity comprises a glycogen branching enzyme (GLYBE).

In another aspect, the present invention relates to a transformed plant as described herein, wherein said heterologous GBE activity comprises a glycogen branching enzyme (GLYBE), and wherein said GLYBE is obtainable from a bacterium.

In another aspect, the present invention relates to a transformed plant as described herein, wherein said heterologous GBE activity comprises a glycogen branching enzyme (GLYBE), and wherein said GLYBE is obtainable from the bacterium Escherichia coli.

In another aspect, the present invention relates to a method for producing starch with modified characteristics, said method comprising; providing a plant having reduced endogenous GBE activity, and having heterologous GBE activity, propagating said plant, and preparing starch from said plant.

In another aspect, the present invention relates to starch obtainable from a transformed plant as described herein.

In another aspect, the present invention relates to starch obtainable from a transformed plant as described herein, wherein said starch comprises at least one starch species having a branching pattern not naturally found in a wild-type parent plant. Preferably said starch comprises starch from a particular tissue, wherein at least one starch species from said tissue has a branching pattern not naturally found in a comparable tissue of a wild-type parent plant.

For ease of reference, these and further aspects of the present invention are now discussed under appropriate section headings. However, the teachings under each section are not necessarily limited to each particular section.

Typically, the term ‘altered’ may mean ‘modified’ as explained above, such as reduced.

### Preferable Aspects

As mentioned, the construct and/or the vector of the present invention may include a transcriptional initiation region which may provide for regulated or constitutive expression. Any suitable promoter may be used for the transcriptional initiation region, such as a tissue specific promoter. Examples of suitable promoters include the patatin promoter or the E35S promoter or the GBSS promoter. According to one aspect, preferably the promoter is the GBSS promoter.

GBE activity may be reduced using one or more appropriate nucleotide sequences relating to an GBE, such as an antisense GBE sequence or a part thereof and/or a sense GBE sequence or a part thereof. Examples of such sequences include one or more exons (or part thereof and/or one or more introns (or part thereof). An organism can be transformed with these sequences by delivering those sequences on the same or different constructs (e.g., vectors).

Preferably, however, GBE activity is reduced using antisense SBE I expression, or antisense SBE II expression, or both antisense SBE I and antisense SBE II expression.

More preferably, GBE activity is reduced using both antisense SBE I and antisense SBE II expression.

### Advantages

A key advantage of the present invention is that it provides a method for preparing modified starches that is not dependent on the need for post-harvest modification of starches. Thus the method of the present invention obviates the need for the use of hazardous chemicals that are normally used in the post-harvest modification of starches.

In addition, the present invention provides inter alia genetically modified plants which are capable of producing modified and/or novel and/or improved starches whose properties would satisfy various industrial requirements.

An other key advantage of the present invention is that it provides a method that may more reliably and/or more efficiently and/or more specifically affect enzymatic activity when compared to the known methods of affecting enzymatic activity.

Thus, the present invention provides a method of preparing particular starches in plants which could replace post-harvest modified starches.

Also, the present invention provides a method that enables modified starches to be prepared by a method that can have a less detrimental effect on the environment than the known post-harvest modification methods which are dependent on the use of hazardous chemicals and large quantities of energy.

### Starch

Starch may occur in various different types. Normal potato starch refers to starch from organisms such as potatoes which do not have a modified GBE activity. Modified starch refers to starch from organisms such as potatoes according to the present invention which have an modified GBE activity.

High amylose starch is an example of a modified starch. High amylose starch has an modified branching pattern. High amylose starch is produced when GBE activity is reduced. High amylose starch has a medium branched character. Floridean type starch is an example of a modified starch. Floridean type starch is produced when GBE activity is reduced, and a heterologous GBE (SBE) activity is present. Glycogen type starch is an example of a modified starch. Glycogen type starch is produced when GBE activity is reduced, and a heterologous GBE activity is present such as a glycogen branching enzyme (a GLYBE).

Starch types may be monitored microscopically, or by a simple viscosity measurement, or by gel filtration, or by amylose determination for example using iodine. Monitoring of starch types may be facilitated by the use of commonly available analytical devices such as the Rapid Visco Analyser (viscosity measurement), or the HPAEC Dionix (high performance anion exchange chromatography), or any other suitable device. Starch types may also be monitored using Nuclear Magnetic Resonance (NMR), preferably solid state NMR. Starch types may also be monitored using mass spectrometry (MS). Starch types may also be monitored using any suitable assay known to those skilled in the art.

### Glucan Branching Enzyme (“GBE”)

GBEs include starch branching enzyme(s) (SBEs), as well as glycogen branching enzyme(s) (GBEs). The term GBE as used herein relates to any glucan branching enzyme and therefore includes starch branching enzyme as well as glycogen branching enzyme (GLYBE) and similar enzymes.

### Starch Branching Enzyme (“SBE”)

SBE catalyses the formation of branch points in the amylopectin (linear or branched glucans) molecule by adding α-1,4 glucans through α-1,6-glucosidic branching linkages. The term SBE includes SBE I, SBE II as well as similar or related enzymes.

### Glycogen Branching Enzyme (GLYBE)

Glycogen branching enzyme (GLYBE) is involved in glycogen metabolism. The term GLYBE includes similar or related enzymes.

### Nucleotide Sequences

The term “nucleotide” in relation to the present invention includes DNA and RNA. Preferably it means DNA, more preferably DNA prepared by use of recombinant DNA techniques.

The term “intron” is used in its normal sense as meaning a segment of nucleotides, usually DNA, that does not encode part or all of an expressed protein or enzyme.

The term “exon” is used in its normal sense as meaning a segment of nucleotides, usually DNA, encoding part or all of an expressed protein or enzyme.

Thus, the term “intron” refers to gene regions that are transcribed into RNA molecules, but which are spliced out of the RNA before the RNA is translated into a protein. In contrast, the term “exon” refers to gene regions that are transcribed into RNA and subsequently translated into proteins.

The terms “variant” or “homologue” or “fragment” in relation to the nucleotide sequence of the present invention include any substitution of, variation of, modification of, replacement of, deletion of or addition of one (or more) nucleic acid from or to the respective nucleotide sequence providing the resultant nucleotide sequence can affect enzyme activity in an organism, such as a plant, or cell or tissue thereof, preferably wherein the resultant nucleotide sequence has at least the same effect as any one of the antisense sequences described herein. In particular, the term “homologue” covers homology with respect to similarity of structure and/or similarity of function providing the resultant nucleotide sequence has the ability to affect enzymatic activity in accordance with the present invention. With respect to sequence homology (i.e. similarity), preferably there is more than 80% homology, more preferably at least 85% homology, more preferably at least 90% homology, even more preferably at least 95% homology, more preferably at least 98% homology. The above terms are also synonymous with allelic variations of the sequences.

Likewise, the terms “variant” or “homologue” or “fragment” in relation to a promoter of the present invention include any substitution of, variation of, modification of, replacement of, deletion of or addition of one (or more) nucleic acid from or to the respective promoter sequence providing the resultant promoter sequence allows expression of a gene of interest (GOI), preferably wherein the resultant promoter sequence has at least the same effect as those described herein. In particular, the term “homologue” covers homology with respect to similarity of structure and/or similarity of function providing the resultant promoter sequence has the ability to allow for expression of a GOI, such as a nucleotide sequence encoding a GBE, or an antisense fragment thereof according to the present invention. With respect to sequence homology (i.e. similarity), preferably there is more than 80% homology, more preferably at least 85% homology, more preferably at least 90% homology, even more preferably at least 95% homology, more preferably at least 98% homology. The above terms are also synonymous with allelic variations of the sequences.

The term “antisense” means a nucleotide sequence that is complementary to, and can therefore hybridize with, any one or all of the sequences of the present invention, including partial sequences thereof. Preferably, the antisense nucleic acids according to the present invention are not complementary to intron sequence(s). Unless otherwise stated, the term ‘antisense sequence’ as used herein means a nucleotide sequence that is antisense to at least part of an GBE exon.

According to the present invention, the antisense nucleic acid is preferably complementary to an entire exon of the gene to be inhibited. However, in some circumstances, partial antisense sequences may be used (i.e. sequences that are not or do not comprise the full complementary sequence) provided that the partial sequences affect enzymatic activity. Suitable examples of partial sequences include sequences that are shorter than any one of the full sequences shown in the sequences disclosed herein, but which comprise nucleotides that are at least antisense to sense sequences found in respective exon or exons. Such partial sequences may be linked, fused, joined, concatenated or otherwise associated. Such sequences may comprise noncontiguous sections of one or more exons, such that for example one such sequence might comprise the end of one exon, linked with the beginning of another, discrete exon to form a single antisense nucleic acid, contigous or otherwise.

With regard to particular aspects of the present invention (i.e. specifically affecting GBE activity), the nucleotide sequences of the present invention may comprise one or more sense or antisense exon sequences of a SBE gene, including complete or partial sequences thereof, providing the nucleotide sequences can affect GBE activity, preferably wherein the nucleotide sequences reduce or eliminate said GBE activity. Preferably, the nucleotide sequences of aspects of the present invention do not comprise an antisense intron sequence.

### Vectors

The term “vector” includes an expression vector and a transformation vector. The term “expression vector” means a construct capable of in vivo or in vitro expression. The term ‘transformation vector’ means a construct capable of being transferred from one species to another—such as from an E. Coli plasmid to a fungus or a plant cell, or from an Agrobacterium to a plant cell.

The term “construct”—which is synonymous with terms such as “conjugate”, “cassette” and “hybrid”—in relation to the antisense nucleotide sequence aspect of the present invention includes the nucleotide sequence according to the present invention directly or indirectly attached to a promoter. An example of an indirect attachment is the provision of a suitable spacer group such as an intron sequence, such as the Sh1-intron or the ADH intron, intermediate the promoter and the nucleotide sequence of the present invention. The same is true for the term “fused” in relation to the present invention which includes direct or indirect attachment. The terms do not cover the natural combination of the wild type SBE gene when associated with the wild type SBE gene promoter in their natural environment.

The construct may even contain or express a marker which allows for the selection of the genetic construct in, for example, a plant cell into which it has been transferred. Various markers exist which may be used in, for example, plants—such as mannose. Other examples of markers include those that provide for antibiotic resistance—e.g. resistance to G418, hygromycin, bleomycin, kanamycin and gentamycin.

### Promoters

The construct of the present invention preferably comprises a promoter. The term “promoter” is used in the normal sense of the word in the art, e.g. an RNA polymerase binding site according to the Jacob-Monod theory of gene expression. Examples of suitable promoters are those that can direct efficient expression of the nucleotide sequence(s) according to the present invention and/or in a specific type of cell. Some examples of tissue specific promoters are disclosed in WO 92/11375.

The promoter could additionally include conserved regions such as a Pribnow Box or a TATA box. The promoters may even contain other sequences to modulate or affect (such as to maintain, enhance, decrease) the levels of expression of the nucleotide sequence of the present invention. Suitable examples of such sequences include the Sh1-intron or an ADH intron. Other sequences include inducible elements—such as temperature, chemical, light or stress inducible elements. Also, suitable elements to enhance transcription or translation may be present. An example of the latter element is the TMV 5′ leader sequence (see Sleat Gene 217 [1987] 217-225; and Dawson Plant Mol. Biol. 23 [1993] 97).

### Additional Sequence of Interest

In addition to reducing GBE activity, the present invention also encompasses transformation of the organism with one or more additional sequences of interest (“GOI”). These GOI(s) may be delivered on the same or different constructs.

Typical examples of a GOI include genes encoding for other proteins or enzymes that modify metabolic and catabolic processes. The GOI may code for an agent for introducing or increasing pathogen resistance.

The GOI may even be an antisense construct for modifying the expression of natural transcripts present in the relevant tissues.

The GOI may even code for a protein that is non-natural to the host organism—e.g. a plant. The GOI may code for a compound that is of benefit to animals or humans. For example, the GOI could code for a pharmaceutically active protein or enzyme such as any one of the therapeutic compounds insulin, interferon, human serum albumin, human growth factor and blood clotting factors. The GOI may even code for a protein giving additional nutritional value to a food or feed or crop. Typical examples include plant proteins that can inhibit the formation of anti-nutritive factors and plant proteins that have a more desirable amino acid composition (e.g. a higher lysine content than a non-transgenic plant). The GOI may even code for an enzyme that can be used in food processing such as xylanases and α-galactosidase. The GOI can be a gene encoding for any one of a pest toxin, an antisense transcript such as that for α-amylase, a protease or a glucanase.

In one aspect the GOI can even be a nucleotide sequence according to the present invention but when operatively linked to a different promoter.

The GOI could include a sequence that codes for one or more of a xylanase, an arabinase, an acetyl esterase, a rhamnogalacturonase, a glucanase, a pectinase, a branching enzyme or another carbohydrate modifying enzyme or proteinase. Alternatively, the GOI may be a sequence that is antisense to any of those sequences.

### Transformed Organisms

The term “organism” in relation to the present invention includes any organism that could comprise the nucleotide sequence according to the present invention and/or wherein the nucleotide sequence according to the present invention can be expressed when present in the organism. Preferably the organism is a starch producing organism such as any one of a plant, algae, fungi, yeast and bacteria, as well as cell lines thereof. Preferably the organism is a plant.

The term “parent plant” refers to a plant isogenic with one which gave rise to a particular transformed plant. The parent plant and its transformed derivative(s) will generally differ only in respect of the transgene(s) used in the creation of the transformed plant(s) from the parent plant. A transformed plant may proceed to become a parent plant for a further modified transformed plant.

The term “wild-type” has its normal meaning in the field of genetics, and may include particular cultivars or crop plant genotypes as appropriate which are not modified as taught herein.

If, for example, the organism is a plant then the promoter according to the present invention can be one that affects expression of the nucleotide sequence in any cell, tissue or organ, such as one or more of seed, tuber, stem, sprout, root and leaf tissues, preferably tuber. The terms “cell”, “tissue” and “organ” include cell, tissue and organ per se and when within an organism.

The term “starch producing organism” includes any organism that can biosynthesise starch. Preferably, the starch producing organism is a plant.

The term “plant” as used herein includes any suitable angiosperm, gymnosperm, monocotyledon and dicotyledon. Typical examples of suitable plants include vegetables such as potatoes; cassava; cereals such as wheat, maize, rice and barley; fruit; trees; flowers; and other plant crops. Preferably, the term means “potato”.

The term “transformed organism” in relation to the present invention includes any organism that comprises the nucleotide sequence according to the present invention and/or products obtained therefrom, and/or wherein the nucleotide sequence according to the present invention can be expressed within the organism. Preferably the nucleotide sequence of the present invention is incorporated in the genome of the organism. Preferably the transformed organism is a plant, more preferably a potato.

To prepare the host organism one can use prokaryotic or eukaryotic organisms. Examples of suitable prokaryotic hosts include E. coli and Bacillus subtilis. Teachings on the transformation of prokaryotic hosts is well documented in the art, for example see Sambrook et al (Sambrook et al. in Molecular Cloning: A Laboratory Manual, 2nd edition, 1989, Cold Spring Harbor Laboratory Press).

### Production of Transformed Plants

The basic principle in the construction of genetically modified plants is to insert genetic information in the plant genome so as to obtain a stable maintenance of the inserted genetic material.

Several techniques exist for inserting the genetic information, the two main principles being direct introduction of the genetic information and introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech March/April 1994 17-27).

A variety of transformation vectors are available for plant transformation and the SBE nucleotide sequences of the present invention can be used in conjunction with any such vectors. The selection of vector will depend on the preferred transformation technique and the plant species which is to be transformed. For certain target species, different selectable markers may be preferred.

For Agrobacterium-mediated transformation, binary vectors or vectors carrying at least one T-DNA border sequence are suitable. A number of vectors are available including pBIN19 (Bevan, Nucl. Acids Res. 12: 8711-8721 (1984), the pBI series of vectors, and pCIB10 and derivatives thereof (Rothstein et al. Gene 53: 153-161 (1987); WO 95/33818).

Binary vector constructs prepared for Agrobacterium transformation are introduced into an appropriate strain of Agrobacterium tumefaciens (for example, LBA 4044 or GV 3101) either by triparental mating (Bevan; Nucl. Acids Res. 12: 8711-8721 (1984)) or direct transformation (Hofgen & Willmitzer, Nucl. Acids Res. 16: 9877 (1988)).

For transformation which is not Agrobacterium-mediated (i.e. direct gene transfer), any vector is suitable and linear DNA containing only the construct of interest may be preferred. Direct gene transfer can be undertaken using a single DNA species or multiple DNA species (co-transformation; Schroder et al. Biotechnology 4: 1093-1096 (1986)).

Construction of vectors according to the invention employs conventional ligation techniques. Isolated plasmids or DNA fragments are cleaved, tailored, and religated in the form desired to generate the constructs required. If desired, analysis to confirm correct sequences in the constructed plasmids is performed in a known fashion. Suitable methods for constructing expression vectors, preparing in vitro transcripts, introducing DNA into host cells, and performing analyses for assessing expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately IGBElled probe which may be based on a sequence provided herein. Those skilled in the art will readily envisage how these methods may be modified, if desired.

DNA may be stably incorporated into cells or may be transiently expressed using methods known in the art. Stably transfected cells may be prepared by transfecting cells with an expression vector having a selectable marker gene, and growing the transfected cells under conditions selective for cells expressing the marker gene. To prepare transient transfectants, cells are transfected with a reporter gene to monitor transfection efficiency.

Heterologous DNA may be introduced into plant host cells by any method known in the art, such as electroporation or Agrobacterium tumefaciens mediated transfer. Although specific protocols may vary from species to species, transformation techniques are well known in the art for most commercial plant species.

In the case of dicotyledonous species, Agrobacterium-mediated transformation is generally a preferred technique as it has broad application to many dicotyledons species and is generally very efficient. Agrobacterium-mediated transformation generally involves the co-cultivation of Agrobacterium with explants from the plant and follows procedures and protocols that are known in the art. Transformed tissue is generally regenerated on medium carrying the appropriate selectable marker. Protocols are known in the art for many dicotyledonous crops including (for example) cotton, tomato, canola and oilseed rape, poplar, potato, sunflower, tobacco and soybean (see for example EP 0 317 511, EP 0 249 432, WO 87/07299, U.S. Pat. No. 5,795,855).

In addition to Agrobacterium-mediated transformation, various other techniques can be applied to dicotyledons. These include PEG and electroporation-mediated transformation of protoplasts, and microinjection (see for example Potrykus et al. Mol. Gen. Genet. 199: 169-177 (1985); Reich et al. Biotechnology 4: 1001-1004 (1986); Klein et al. Nature 327: 70-73 (1987)). As with Agrobacterium-mediated transformation, transformed tissue is generally regenerated on medium carrying the appropriate selectable marker using standard techniques known in the art.

Until very recently there were very few reports on successful production of transformed monocotyledon crop plants. Although Agrobacterium-mediated transformation has been applied successfully to monocotyledonous species such as rice and maize and protocols for these approaches are available in the art, the most widely used transformation techniques for monocotyledons remain particle bombardment, and PEG and electroporation-mediated transformation of protoplasts. This relatively slow development within monocots was due to two causes. Firstly, until the early 1980s, efficient regeneration of plants from cultured cells and tissues of monocots had proven very difficult. This problem was ultimately solved by the culture of explants from immature and embryogenic tissue, which retain their morphogenic potential on nutrient media containing plant growth regulators. Secondly, the monocots are not a natural host for Agrobacterium tumefaciens, meaning that the successful developed techniques within the dicots using their natural vector Agrobacterium tumefaciens was unsuccessful for many years in the monocots.

Nevertheless, it is now possible to successfully transform and produce fertile transformed plants of maize using methods such as: (1) Silicon Carbide Whiskers; (2) Particle Bombardment; (3) DNA Uptake by PEG treated protoplast; or (4) DNA Uptake in Electroporation of Tissue. Each of these methods—which are reviewed by Thompson (1995 Euphtytica 85 pp 75-80)—may be used to prepare inter alia transformed plants according to the present invention.

In particular, the particle Gun method has been successfully used for the transformation of monocots. However, EP-A-0604662 reports on a different method of transforming monocotyledons. The method comprises transforming cultured tissues of a monocotyledon under or after dedifferentiation with Agrobacterium containing a super binary vector as a selection means a hygromycin-resistant gene was used. Production of transformed calli and plant was demonstrated using the hygromycin selection. This method may be used to prepare inter alia transformed plants according to the present invention.

In the case of maize, Gordon-Kamm et al. (Plant Cell 2: 603-618 (1990)), Fromm et al. (Biotechnology 8: 833-839 (1990) and Koziel et al. (Biotechnology 11: 194-200 (1993)) have published techniques for transformation using particle bombardment.

In the case of rice, protoplast-mediated transformation for both Japonica- and Indica-types has been described (Zhang et al. Plant Cell Rep. 7: 379-384 (1988); Shimamoto et al. Nature 338: 274-277; Datta et al. Biotechnology 8: 736-740 (1990)) and both types are also routinely transformable using particle bombardment (Christou et al. Biotechnology 9: 957-962 (1991)).

In the case of wheat, transformation by particle bombardment has been described for both type C long-term regenerable callus (Vasil et al. Biotechnology 10: 667-674 (1992)) and immature embryos and immature embryo-derived callus (Vasil et al. Biotechnology 11: 1553-1558 (1993); Weeks et al. Plant Physiol. 102: 1077-1084 (1993)). A further technique is described in published patent applications WO 94/13822 and WO 95/33818.

### Selectable Markers

Transformation of plant cells is normally undertaken with a selectable marker which may provide resistance to an antibiotic or to a herbicide. Selectable markers that are routinely used in transformation include the nptl gene which confers resistance to kanamycin (Messing & Vierra Gene 19: 259-268 (1982); Bevan et al. Nature 304: 184-187 (1983)), the bar gene which confers resistance to the herbicide phosphinothricin (White et al. Nucl. Acids Res. 18: 1062 (1990); Spencer et al. Theor. Appl. Genet. 79: 625-631 (1990)), the hph gene which confers resistance to the antibiotic hygromycin (Blochlinger & Diggelmann Mol. Cell Biol. 4: 2929-2931 (1984)), and the dhfr gene which confers resistance to methotrexate (Bourouis et al. EMBO J 2: 1099-1104 (1983)). More recently, a number of selection systems have been developed which do not rely of selection for resistance to antibiotic or herbicide. These include the inducible isopentyl transferase system described by Kunkel et al. (Nature Biotechnology 17: 916-919 (1999), or the mannose system or xylose system (described in more detail below).

### Codon Preference

The nucleic acid constructs of the invention are suitable for expression in a variety of different organisms. However, to enhance the efficiency of expression it may be necessary to modify the nucleotide sequence encoding the GBE(s) to account for different frequencies of codon usage in different host organisms. Hence it is preferable that the sequences to be introduced into organisms, such as plants, conform to preferred usage of codons in the host organism.

In general, high expression in plants is best achieved from codon sequences that have a GC content of at least 35% and preferably more than 45%. This is thought to be because the existence of ATTTA motifs destabilize messenger RNAs and the existence of AATAAA motifs may cause inappropriate polyadenylation, resulting in truncation of transcription. Murray et al. (Nucl. Acids Res. 17: 477-498 (1989)) have shown that even within plants, monocotyledonous and dicotyledonous species have differing preferences for codon usage, with monocotyledonous species generally preferring GC richer sequences. Thus, in order to achieve optimal high level expression in plants, gene sequences can be altered to accommodate such preferences in codon usage in such a manner that the codons encoded by the DNA are not changed.

Plants also have a preference for certain nucleotides adjacent to the ATG encoding the initiating methionine and for most efficient translation, these nucleotides may be modified. To facilitate translation in plant cells, it is preferable to insert, immediately upstream of the ATG representing the initiating methionine of the gene to be expressed, a “plant translational initiation context sequence”. A variety of sequences can be inserted at this position. These include the sequence the sequence 5′-AAGGAGATATAACAATG-3′ (Prasher et al. Gene 111: 229-233 (1992); Chalfie et al. Science 263: 802-805 (1992)), the sequence 5′-GTCGACCATG-3′ (Clontech 1993/1994 catalog, page 210), and the sequence 5′-TAAACAATG-3′ (Joshi et al. Nucl. Acids Res. 15: 6643-6653 (1987)). For any particular plant species, a survey of natural sequences available in any databank (e.g. GenBank) can be undertaken to determine preferred “plant translational initiation context sequences” on a species-by-species basis.

Any changes that are made to the coding sequence can be made using techniques that are well known in the art and include site directed mutagenesis, PCR, and synthetic gene construction. Well known protocols for transient expression in plants can be used to check the expression of modified genes before their transfer to plants by transformation.

Thus, in summary, the present invention relates to a transformed organism, having a reduced endogenous glucan branching enzyme (GBE) activity, and having a heterologous glucan branching enzyme activity. Preferably the transformed organism is a transformed plant.

In a preferred aspect, the present invention relates to a transformed organism having a reduced endogenous glucan branching enzyme (GBE) activity, and having a heterologous glucan branching enzyme activity, wherein said reduced GBE activity is effected via expression of a nucleotide sequence that is antisense to at least part of an GBE exon.

In a more preferred aspect, the present invention relates to a transformed organism having a reduced endogenous glucan branching enzyme (GBE) activity, and having a heterologous glucan branching enzyme activity, wherein said reduced GBE activity is effected via expression of a nucleotide sequence that is antisense to at least part of an GBE exon, and wherein said organism is a plant.

The present invention will now be described, by way of example only, in which reference will be made to the following figures.

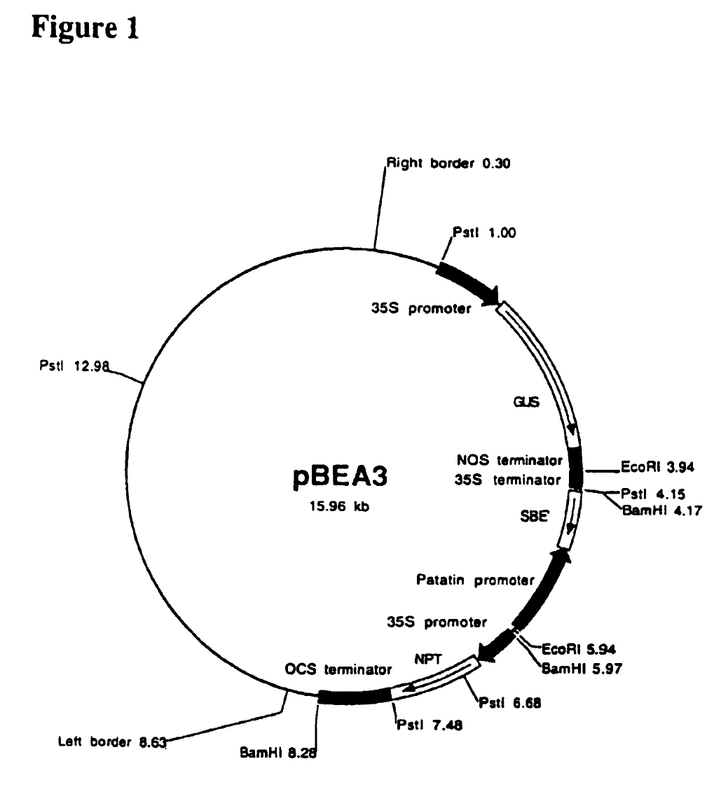

FIG. 1, which shows a plasmid.

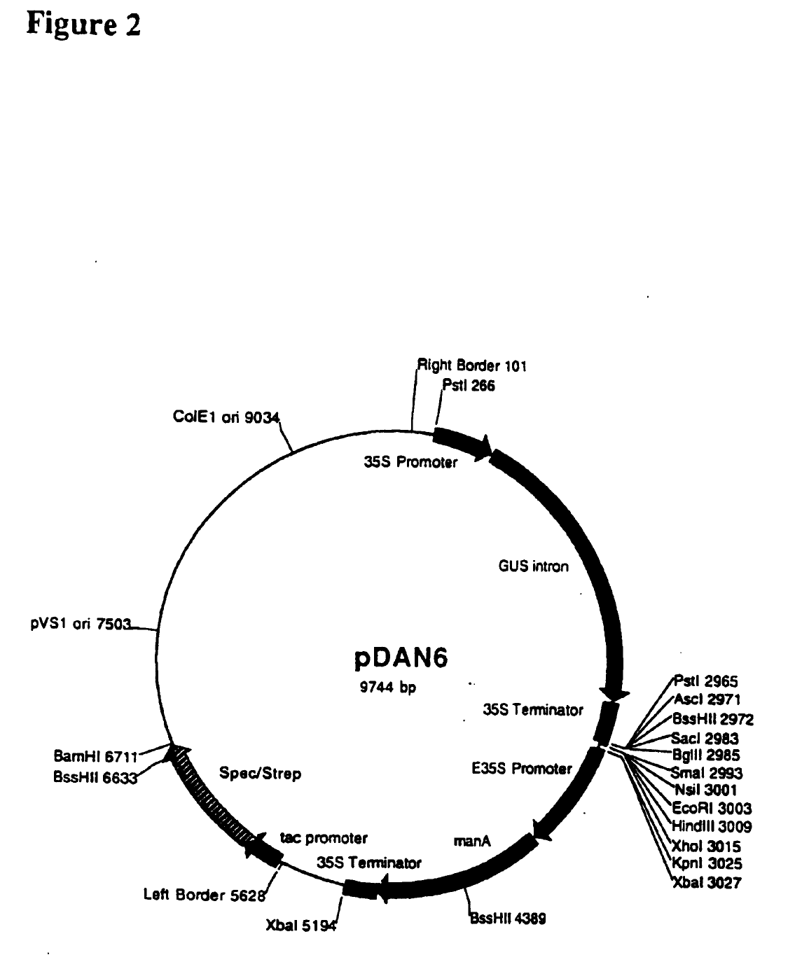

FIG. 2, which shows a plasmid.

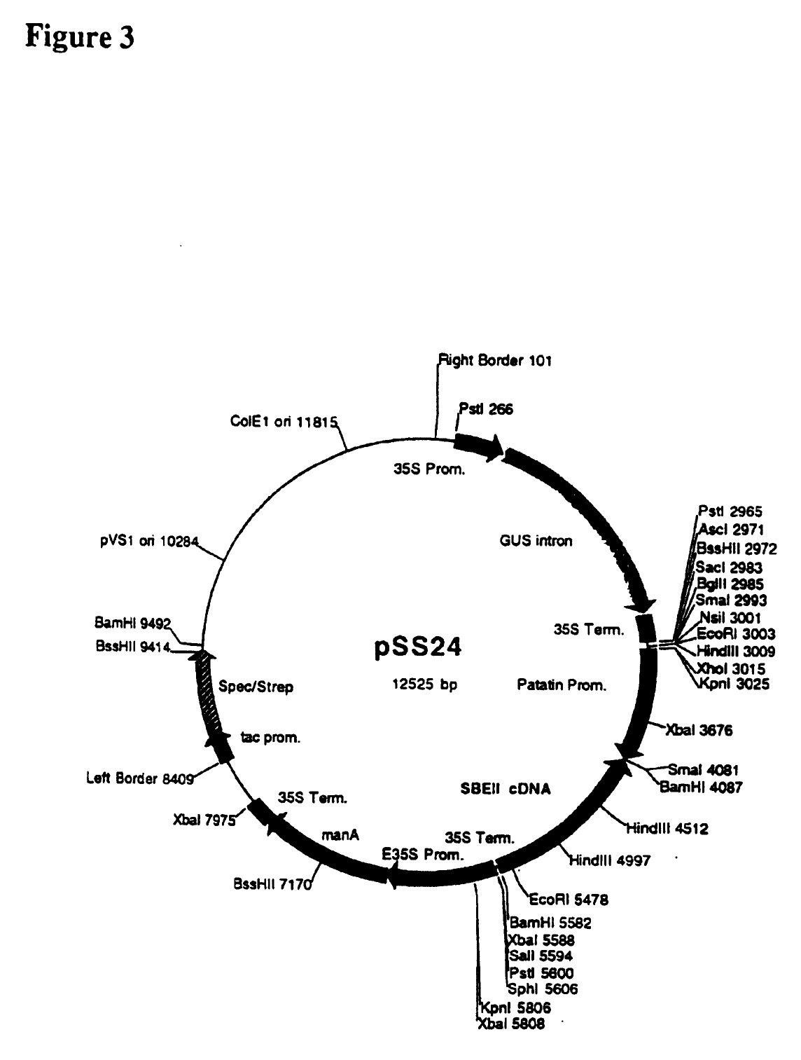

FIG. 3, which shows a plasmid.

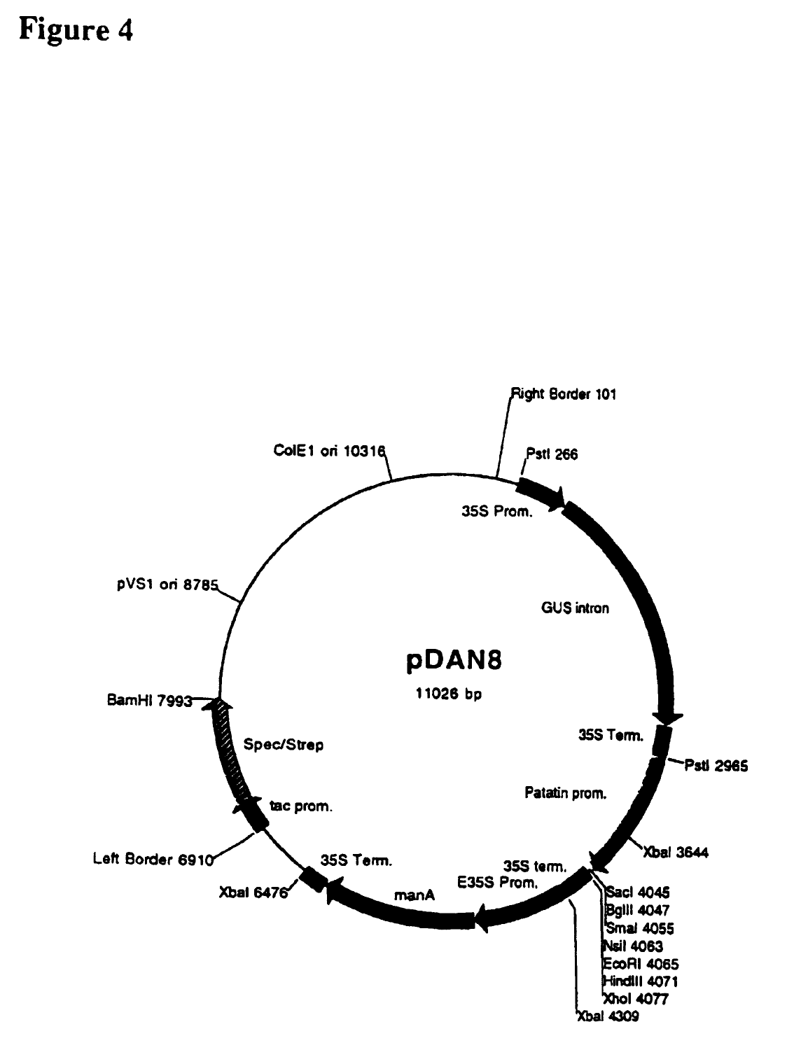

FIG. 4, which shows a plasmid.

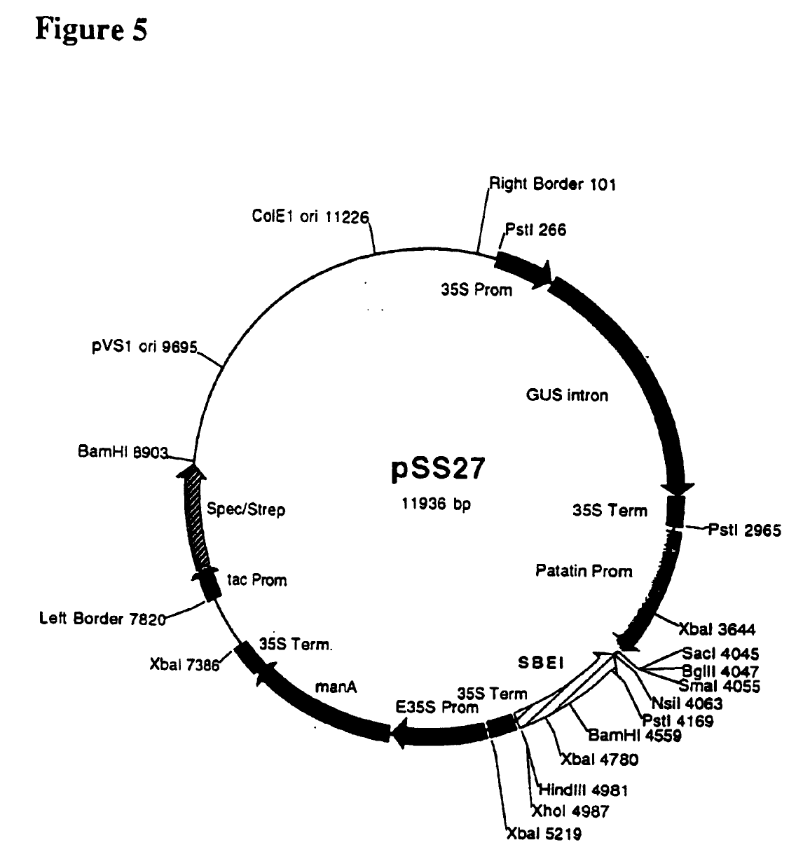

FIG. 5, which shows a plasmid.

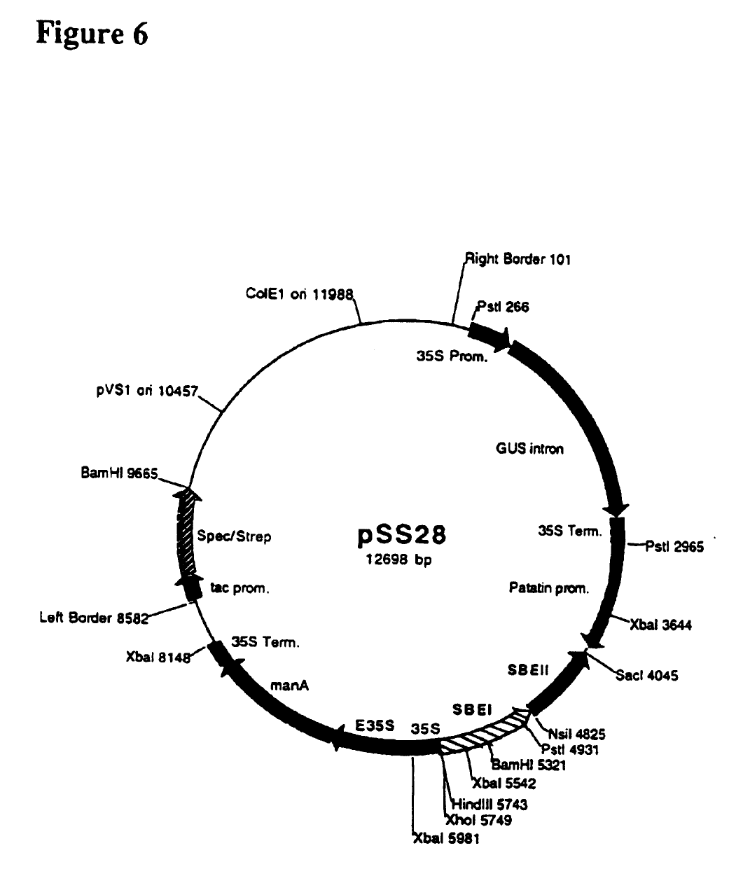

FIG. 6, which shows a plasmid.

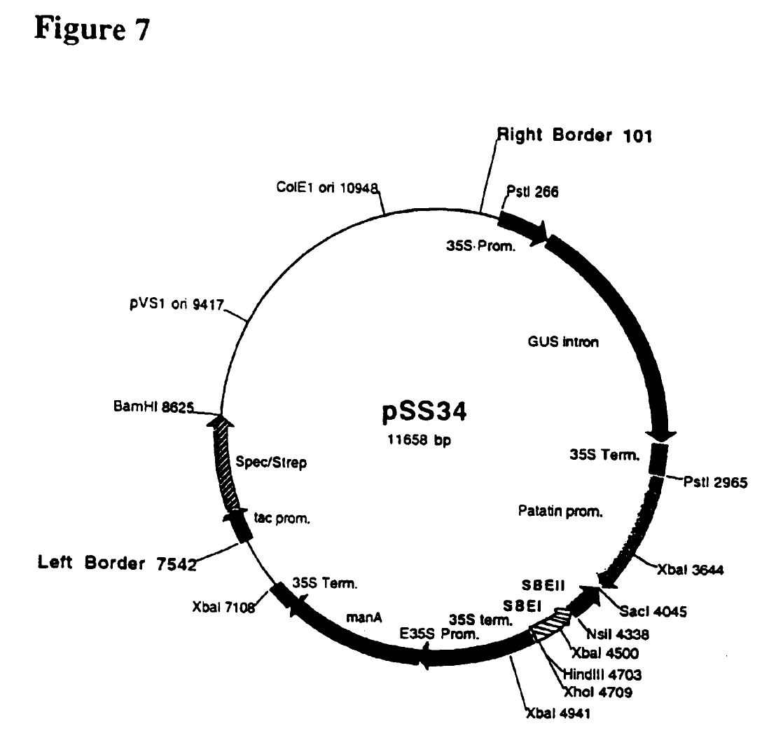

FIG. 7, which shows a plasmid.

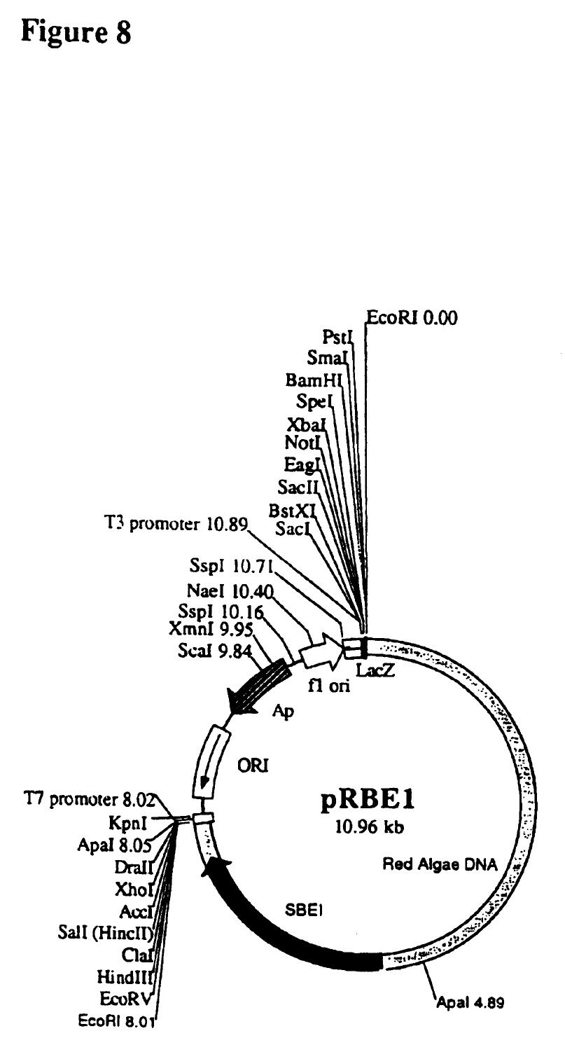

FIG. 8, which shows a plasmid.

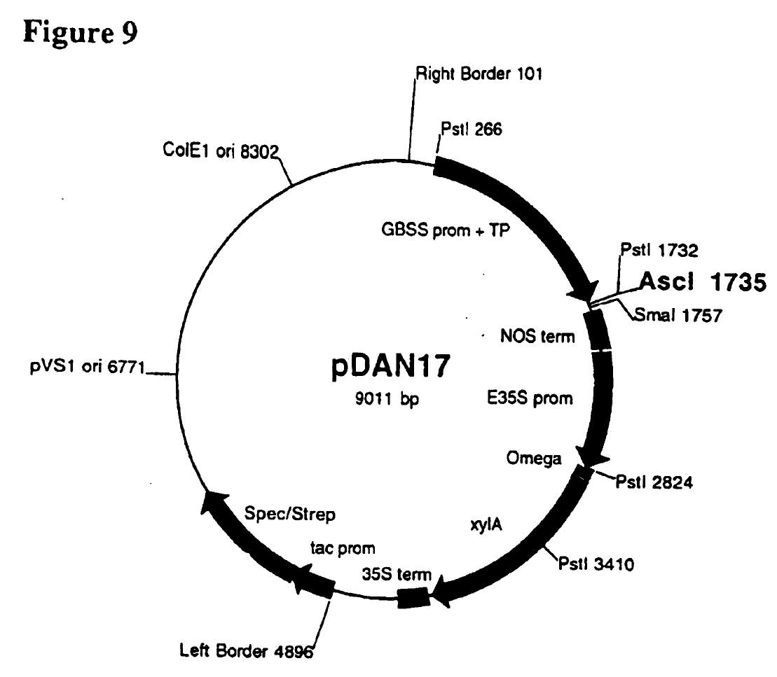

FIG. 9, which shows a plasmid.

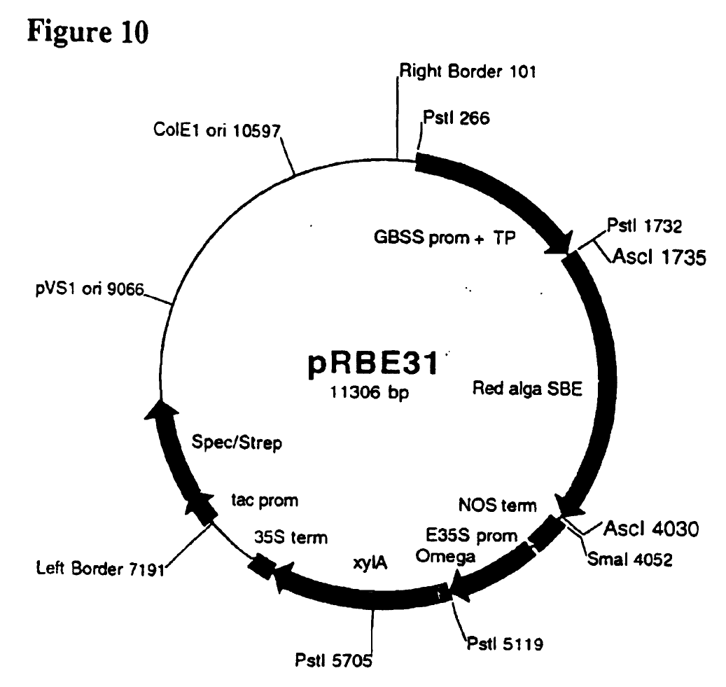

FIG. 10, which shows a plasmid.

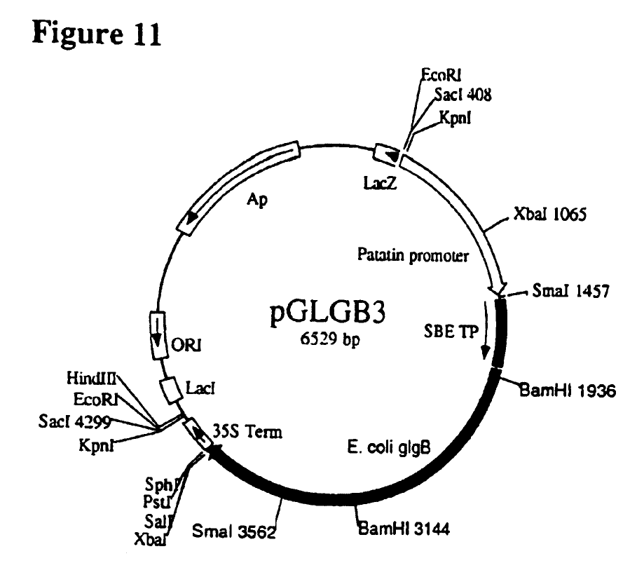

FIG. 11, which shows a plasmid.

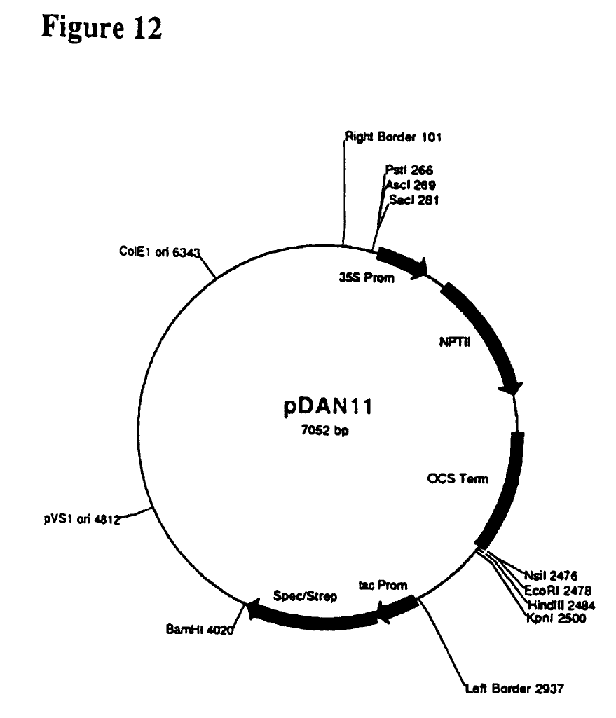

FIG. 12, which shows a plasmid.

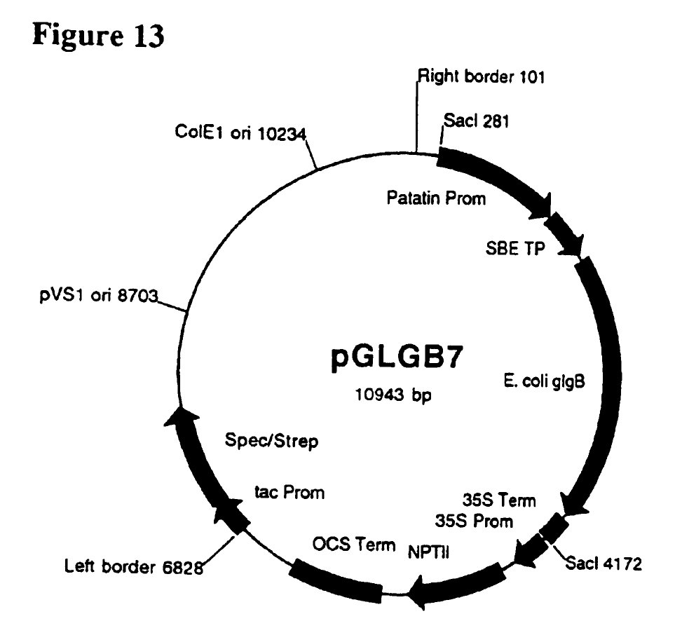

FIG. 13, which shows a plasmid.

### EXAMPLES

### Example 1

### Reduction of Endogenous Starch Branching Enzyme Activity

Glucan branching enzyme (GBE) activity is reduced in plants by expressing antisense GBE nucleic acids in said plants. This is accomplished using SBE I antisense expression, or using SBE II antisense expression, or using SBE I and SBE II antisense expression, as discussed herein.

Reduction of starch branching enzyme activity by antisense SBE I expression.

Nucleic acid constructs for the expression of antisense SBE are prepared.

A 692 bp EcoRI fragment from the 5′ end of a potato starch branching enzyme I (SBEI) cDNA (Poulsen and Kreiberg (1993) Plant physiol 102:1053-1054) is inserted in antisense orientation after a patatin class I promoter in plasmid pPATA1 (see WO94/24292). From the resulting plasmid, pGBE3, the antisense SBEI cassette is isolated as a 1996 bp EcoRI restriction fragment, which is inserted into the plant transformation vector pBKL4 (see WO94/24292) yielding plasmid pBEA3 (shown in FIG. 1).

Potato plants are transformed using kanamycin resistance as the selectable marker, according to techniques well known in the art, and as discussed herein, and transformed plants are isolated.

These transformed plants have reduced GBE activity.

Reduction of starch branching enzyme activity by antisense SBE II expression.

Nucleic acid constructs for the expression of antisense SBE are prepared.

A potato SBE II cDNA clone is obtained by RT-PCR from potato sprout total RNA using the primers 5′-TCA GCA GTA ATG GTG ATC GGA GG-3′ and 5′-CAC AAG TTC GTT CAT TCT TCT TCT AC-3′ and a Titan One Tube RT-PCR Kit (Boehringer Mannheim). The RT-PCR program is as follows: 50° C. for 30 minutes, 95° C. for 4 minutes, 35 cycles of 92° C. for 30 seconds, 62° C. for 45 seconds, and 68° C. for 2 minutes, and a final elongation step of 68° C. for 7 minutes. The resulting 2.6 kb cDNA fragment is inserted into pCR2.1-TOPO to give plasmid pSS19.1. The nucleotide sequence of the SBE II cDNA is shown in SEQ. ID. NO. 1.

Using plasmid pSS19.1 as DNA template a 1495 bp SBE II DNA fragment from the 5′ end of the SBE II cDNA is PCR amplified with the primers 5′-CGG GAT CCC GTC AGC AGT AAT GGT GAT CGG AGG-3′ and 5′-CGG GAT CCC GAC CGA TAA TCC GTG GTG AG. This fragment is inserted as a BamHI fragment in an antisense orientation after the patatin promoter in plasmid pPATA1 (see WO94124292) to give plasmid pSS21.

The SBE II antisense cassette from this plasmid pSS21 is isolated as a 2781 bp Kpnl fragment and inserted in the Kpnl site of pDAN6 (FIG. 2) yielding pSS24 (FIG. 3).

Potato plants are transformed using the mannose selection principle (see United States Patent No. 5,767,378).

A number of transformed plants are isolated.

These transformed plants have reduced GBE activity.

Reduction of starch branching enzyme activity by antisense SBE I and antisense SBE II expression.

Nucleic acid constructs for the expression of antisense SBE I and SBE II are prepared.

A 918 bp fragment is amplified from the SBE I cDNA (see above) by PCR using the primers 5′-CCC MG CTT CCC GTC TGT MG CAT CAT TAG TG-3′ and 5′-CCA ATG CAT AGG GCG AGG GTA TTT GAA GTG G-3′. The PCR fragment is digested with NsiI and HindIII and inserted into these sites in pDAN8 (FIG. 4) to give plasmid pSS27 (FIG. 5).

A 5′ end potato SBE II cDNA is obtained by RT-PCR from potato sprout total RNA using the primers 5′-TTG ATG GGG CCT TGA ACT CAG C-3′ and 5′-ACC CTC ATA CTT GTC MT TGC CTC-3′ and a Titan One Tube RT-PCR Kit (Boehringer Mannheim). The RT-PCR program is as follows: 50° C. for 30 minutes, 95° C. for 5 minutes, 35 cycles of 92° C. for 45 seconds, 66° C. for 45 seconds, an 68° C. for 1 minutes, and a final elongation step of 68° C. for 7 minutes. Two slightly different 0.7 kb cDNA fragments are obtained and inserted in pCR2.1-TOPO to give plasmids pSS25 and pSS26. The nucleotide sequences of these 5′ end SBEII cDNAs are shown in SEQ. ID. NO. 2 and SEQ. ID. NO. 3.

Using plasmid pSS25 as DNA template a SBE II DNA fragment with SacI and NsiI restriction sites at the ends is generated by PCR using the primers 5′-TAG GCG AGC TCA CCC TCA TAC TTG TCA ATT GCC TC-3′ and 5′-CCA ATG CAT TTG ATG GGG CCT TGA ACT CA-3′. The SacI and NsiI restriction fragment is then inserted into pSS27 (described above-see FIG. 5) yielding pSS28 (FIG. 6).

This plasmid is used for inhibition of SBE I and SBE II expression in potato tubers by transforming potato plants using the mannose selection principle (see U.S. Pat. No. 5,767,378).

Another plasmid (pS34) useful for inhibition of SBE I and SBE II expression in potato tubers is also constructed. This plasmid is similar to plasmid pSS28 (described above), but includes only 345 bp of 5′ end SBE I cDNA and 277 bp of 5′ end SBE II cDNA. pSS34 is constructed by production of a SBE I cDNA fragment by PCR using the primers 5′-CCC MG CTT CCC GTC TGT MG CAT CAT TAG TG-3′ and 5′-CCA ATG CAT AGC GGA MT AGC TGA ACT GTG CTT CAT C-3′.

The resulting NsiI and HindIII restriction fragment is inserted in pDAN8 digested with the same restriction enzymes yielding pSS31. Similarly, the SBE II cDNA fragment is generated using the primers 5′-CCA ATG CAT TTG ATG GGG CCT TGA ACT CA-3′ and 5′-TAG GCG AGC TCG CCA AGA TGT GM AGA GAG TGC-3′ and the resulting PCR fragment is inserted as a SacI-NsiI restriction fragment in pSS31 to give pSS34 (FIG. 7).

Potato plants are transformed using the mannose selection principle (see U.S. Pat. No. 5,767,378).

A number of transformed plants are isolated.

These transformed plants have reduced GBE activity.

### EXAMPLE 2

### Cloning of Genes Encoding Glucan Branching Enzymes (GBEs)

GBE genes are isolated for heterologous expression in organisms according to the present invention, such as plants.

In this Example, an GBE gene is isolated from the red alga Gracilaria lemenefformis. In this Example, the GBE gene isolated is a SBE gene.

A pair of degenerated PCR primers (5′-(TC)T(GATC)ATGGC(GATC)AT(ATC)ATGGA(GA)CA-3′ and 5′-CC(GA)TC(GA)AA(GATC)CG(GA)M(GATC)CC(GA)TC-3′) are designed from two conserved amino acid motifs in starch branching enzymes (SBEs). These motifs are the LMAIMEH and FDGFRFDG, found in the central region of the enzymes.

The PCR primers and chromosomal Gracilana lemeneiformis DNA are used in a PCR amplification with Taq DNA polymerase. The PCR program is as follows: denaturation at 94° C. for 2 minutes, 30 cycles of denaturation at 94° C. for 30 sec, annealing at 42° C. for 1 minutes, and extension at 72° C. for 2 minutes, followed by one extension step at 72° C. for 7 minutes.

The fragments obtained are inserted into pCR-SCRIPT SK (+) (Stratagene, USA) and sequenced. One of the fragments is found to have the desired branching enzyme sequences (SEQ. ID. NO. 4). The 352 bp insert is then used as a radioactive DNA hybridisation probe for screening of a genomic Gracilaria lemaneiformis ZAP II library (Bojsen et al. (1999) Biochimica et Biophysica acta 1430:396-402) using standard protocols (Sambrook et al. (1989) Molecular cloning: a laboratory manual, Second edition, Cold Spring Harbor Laboratory, Cold Spring Harbor, New York).

One of the clones obtained is excised in vivo using the protocols provided by Stratagene (USA) yielding pRBE1 (FIG. 8), which is the pBluescript SK (−) vector containing an 8 kb red algae genomic DNA. The branching enzyme gene is located on a 3.5 kb ApaI DNA fragment which is sequenced (shown as SEQ. ID. NO. 5).

The nucleotide sequence reveals an open reading which encodes a 760 amino acid polypeptide with high similarity to plant starch branching enzymes and eukaryotic glycogen branching enzymes.

Thus, an alignment of the proposed red algae branching enzyme with the mature forms of potato starch branching enzyme I and potato starch branching enzyme II reveals 375 identites (44%) and 367 identites (42%), respectively. This strongly suggests that the cloned gene is a red algae branching enzyme. A comparison with the E. coli glycogen branching enzyme shows that the two enzymes have 142 identities (16%). Thus, the cloned algae branching enzyme is closely related to the plant starch branching enzymes. Since the algae enzyme lacks the 11 amino acid motif found in the proposed 8^{th} loop of the class A plant starch branching enzymes (Burton et al. (1995) The Plant Journal 7(1), 3-15) the enzyme is most likely a class B type of branching enzyme.

Recently, a gene for a starch branching enzyme was isolated from the red alga Gracilaria gracilis (Lluisma & Ragan (1998) Curr Genet 34:105-111). The G. lemaneifornis and G. gracilis genes show significant sequence similarity in the coding regions.

A comparison of the encoded proteins shows that the two branching enzymes have 616 identical amino acids (80% similarity/identity). The differences between the two enzymes are mainly found in the N- and C- termini. Thus, the two branching enzymes may be two isoforms with differing enzymatic activities.

In this way, genes encoding GBEs may be isolated from a variety of sources for use in the present invention.

### EXAMPLE 3

### Heterologous Expression of Starch Branching Enzymes

The present invention provides for the heterologous expression in plants of GBEs from various different species.

Heterologous expression of algal GBEs In plants

In this Example, the heterologous expression of algal GBE in a higher plant is disclosed. In this example, the algal GBE is algal SBE.

A nucleic acid construct for expression of a starch branching enzyme from the red alga Gracilaria lemaneiformis in potato tubers is produced as follows:

The gene encoding the Gracilaria lemaneiformis branching enzyme is amplified by PCR using the primers 5′-GGC GCG CCG GGC TCG GM GAC CC-3′ and GGC GCG CCT CAC ACA GCT TCC TTC TG-3′ with pRBE1 as DNA template.

The PCR fragment is inserted in pCR2.1-TOPO (Invitrogen, The Netherlands) yielding pRBE28. From this plasmid the starch branching enzyme gene is isolated as an AscI restriction fragment and inserted in the AscI site of the plant transformation vector pDAN17 (FIG. 9) after the GBSS promoter and GBSS transit peptide resulting in pRBE31 (FIG. 10).

This plasmid is used for transformation of potato plants using the xylose selection principle (Haldrup et al. (1998) Plant Mol Biol 37:287-296).

A number of transformed plants are isolated which heterologously express the Gracilaria lemaneiformis SBE.

These transformed plants have modified GBE activity.

Heterologous expression of bacterial GBEs in plants

In this Example, the heterologous expression of bacterial GBE in a higher plant is disclosed.

A nucleic acid construct for expression of an glucan branching enzyme (a glycogen branching enzyme (GLYBE)) from the bacterium Escherichia coli in potato tubers is produced as follows:

The E. coli gIgB gene is amplified from λ-phage 616 from the Kohara E. coli λ-phage collection (Kohara et al. (1987) Cell 50:495-508) using the primers: 5′-GAA GAT CTA TCC GAT CGT ATC GAT AGA GAC-3′ and 5′-GAA GAT CTA TCA TTC TGC CTC CCG MC C-3′. The PCR program is: 95° C. for 5 minutes, 25 cycles of 95° C. for 1 minutes, 55° C. for 1 minutes, and 75° C. for 3 minutes, followed by a 10 minutes elongation step at 75° C. Pfu DNA polymerase (Stratagene, USA) is used in the PCR.

The 2184 bp PCR product is digested with BglII and inserted in pBETP5 (see WO 94/24292), and cut with BamHI to give pGLGB3 (FIG. 11). The 3891 bp SacI fragment from pGLGB3 is then inserted in the SacI site of pDAN11 (FIG. 12) yielding pGLGB7 (FIG. 13).

This plasmid is used for transformation of potato plants using the appropriate selectable marker as described above.

A number of transformed plants are isolated which heterologously express the Escherichia coli GBE glycogen branching enzyme (GLYBE).

These transformed plants have modified GBE activity.

### EXAMPLE 4

### Production of Floridean Starch Types in Potatoes

The invention provides for the production in certain plants of starch types which are not naturally found in such plants.

In this Example, the invention provides for the production of floridean starch types in potatoes.

This is accomplished in potato plants by reducing endogenous GBE activity via the use of antisense construct expression, and by providing modified GBE activity via heterologous expression of algal GBE (in this example, the algal GBE is algal SBE) as follows.

Repression of endogenous SBE I and SBE II expression by transformation of potato plants with plasmid pSS28 or plasmid pSS34 as described in Example 1.

Mannose selection is used in the transformation of potato plants, and transformed plants are isolated.

These transformed plants have reduced GBE activity.

A nucleic acid construct for expression of a starch branching enzyme from the red alga Gracilaria lemaneiformis is produced as in Example 3.

This construct (pRBE31) is used for transformation of the transformed potato plants with reduced GBE activity using the xylose selection principle (Haldrup et al. (1998) Plant Mol Biol 37:287-296).

A number of transformed plants are isolated which heterologously express the Gracilaria lemaneiformis SBE.

These transformed plants have modified GBE activity.

Starch is prepared from these transformed plants having modified GBE activity, and is found to comprise floridean starch types.

### EXAMPLE 5

### Production of “Floridean Starch Types” in Potato S

The invention provides for the production in certain plants of starch types which are not naturally found in such plants.

In this Example, the invention provides for the production of floridean starch types in potatoes.

This is accomplished in potato plants by reducing endogenous GBE activity via the use of antisense construct expression, and by providing modified GBE activity via heterologous expression of algal SBE as follows.

First, repression of endogenous SBE II expression is brought about by transformation of potato plants with plasmid pSS24 as described in Example 1.

Mannose selection is used in the transformation of potato plants, and transformed plants are isolated.

These transformed plants have reduced SBE II activity.

Next, repression of endogenous SBE I expression is brought about by transformation of the SBE II repressed plants with plasmid pBEA3 as described in Example 1.

Kanamycin selection is used in the transformation of potato plants, and transformed plants are isolated.

These transformed plants have reduced SBE I activity, and reduced SBE II activity.

A nucleic acid construct for expression of a starch branching enzyme from the red alga Gracilaria lemaneiformis is produced as in Example 3.

This construct (pRBE31) is used for transformation of the transformed potato plants with reduced SBE I and SBE II activity using the xylose selection principle (Haldrup et al. (1998) Plant Mol Biol 37:287-296).

A number of transformed plants are isolated which heterologously express the Gracilaria lemaneifonnis SBE.

These transformed plants have modified GBE activity.

Starch is prepared from these transformed plants having modified GBE activity, and is found to comprise floridean starch types.

### EXAMPLE 6

### Production of “Glycogen-Starch Types” in Potatoes

The invention provides for the production in certain plants of starch types which are not naturally found in such plants.

In this Example, the invention provides for the production of glycogen starch types in potatoes.

This is accomplished in potato plants by reducing endogenous GBE activity via the use of antisense construct expression, and by providing modified GBE activity via heterologous expression of bacterial SBE as follows.

Repression of endogenous SBE I and SBE II expression by transformation of potato plants with plasmid pSS28 or plasmid pSS34 as described in Examples 1 and 4 above.

Mannose selection is used in the transformation of potato plants, and transformed plants are isolated.

These transformed plants have reduced GBE activity.

A nucleic acid construct for expression of a starch branching enzyme (a glycogen branching enzyme (GLYBE)) from the bacterium Escherichia coli is produced as in Example 3.

This construct (pGLGB7—FIG. 13) is used for transformation of the transformed plants having reduced GBE activity, using the appropriate selectable marker (kanamycin) as described above.

A number of transformed plants are isolated which heterologously express the Escherichia coli GBE glycogen branching enzyme (GLYBE).

These transformed plants have modified GBE activity.

Starch is prepared from these transformed plants having modified GBE activity, and is found to comprise glycogen starch types.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A transformed organism, preferably a transformed plant, having a reduced endogenous starch branching enzyme (SBE) activity, and having a heterologous glucan branching enzyme (GBE) activity.

2. A transformed organism, preferably a transformed plant, having a reduced endogenous starch branching enzyme (SBE) activity, and having a heterologous glucan branching enzyme (GBE) activity, wherein said reduced SBE activity is effected via expression of a nucleotide sequence that is antisense to at least part of a SBE exon.

3. A transformed plant according to claim 2 having a reduced endogenous starch branching enzyme (SBE) activity, and having a heterologous glucan branching enzyme (GBE) activity, wherein said SBE exon is selected from the group consisting of SBE I exon(s), SBE II exon(s), or combinations thereof.

4. A transformed plant having a reduced endogenous starch branching enzyme (SBE) activity, and having a heterologous glucan branching enzyme (GBE) activity, wherein said reduced SBE activity is effected via antisense exon expression, and wherein the antisense exon expression system and the heterologous GBE expression system are present as separate nucleic acid constructs.

5. A transformed plant according to any previous claim, wherein said heterologous GBE activity comprises
i) a starch branching enzyme (SBE), or
ii) a glycogen branching enzyme (GLYBE), or
iii) both an SBE and a GLYBE.

6. A transformed plant according to any previous claim, wherein said heterologous GBE activity comprises a SBE obtainable from an alga.

7. A transformed plant according to any previous claim, wherein said heterologous GBE activity comprises a SBE obtainable from a red alga.

8. A transformed plant according to any previous claim, wherein said heterologous GBE activity comprises a SBE obtainable from one or more algae selected from Gracilaria gracilis and Gracilaria lemaneiformis.

9. A transformed plant according to any previous claim, wherein said heterologous GBE activity comprises a glycogen branching enzyme (GLYBE).

10. A transformed plant according to any previous claim, wherein said heterologous GBE activity comprises a GLYBE obtainable from a bacterium.

11. A transformed plant according to any previous claim, wherein said heterologous GBE activity comprises a glycogen branching enzyme (GLYBE), and wherein said GLYBE is obtainable from the bacterium Escherichia coli.

12. A method for producing starch with altered characteristics, said method comprising;
i) providing a plant having reduced endogenous SBE activity, and having heterologous GBE activity,
ii) propagating the plant of (i), and optionally
iii) obtaining starch from said plant.

13. Starch obtainable from a transformed plant according to any previous claim.

14. Starch, wherein said starch comprises at least one starch species having a branching pattern not naturally found in a wild-type parent plant.

15. A nucleic acid contruct system capable of directing the expression of all or part of one or more antisense SBE exon(s) and optionally one or more heterologous GBE(s).

16. A nucleic acid contruct system according to claim 14, wherein the antisense SBE and heterologous GBE elements are present as separate nucleic acid constructs.


SEQUENCE LISTING (30 sequences)

SEQ ID NO: 1 - DNA, 2563 bp, Artificial Sequence
  Description of Artificial Sequence SBEII cDNA
tcagcagtaatggtgatcggaggaatgctaatatttctgtattcttgaaaaaacactctctttcacggaagatcttggctgaaaagtcttcttacaattccgaatcccgaccttctacagttgcagcatcggggaaagtccttgtgcctggaacccagagtgatagctcctcatcctcaacagaccaatttgagttcactgagacatctccagaaaattccccagcatcaactgatgtagatagttcaacaatggaacacgctagccagattaaaactgagaacgatgacgttgagccgtcaagtgatcttacaggaagtgttgaagagctggattttgcttcatcactacaactacaagaaggtggtaaactggaggagtctaaaacattaaatacttttgaagagacaattattgatgaatctgataggatcagagagaggggcatccctccacctggacttggtcagaagatttatgaaatagacccccttttgacaaactatcgtcaacaccttgattacaggtattcacagtacaagaaactgagggaggcaattgacaagtatgagggtggtttggaagctttttctcgtggttatgaaaaaatgggtttcactcgtagtgctacaggtatcacttaccgtgagtgggctcctggtgcccagtcagctgccctcattggagatttcaacaattgggacgcaaatgctgacattatgactcggaatgaatttggtgtctgggagatttttctgccaaataatgtggatggttctcctgcaattcctcatgggtccagagtgaagatacgtatggacactccatcaggtgttaaggattccattcctgcttggatcaactactctttacagcttcctgatgaaattccatataatgggatatattatgatccacccgaagaggagaggtatatcttccaacacccacggccaaagaaaccaaagtcgctgagaatatatgaatctcatattggaatgagtagtccggaacctaaaattaactcatacgtgaattttagagatgaagttcttcctcgcataaaaaagcttgggtacgatgcggtgcaaattatggctattcaagagcattcttattatgctagttttggttatcatgtcacaaatttttttgcaccaagcagccgttttggaactcccgacgaccttaagtctttgattgataaagctcatgagctaggaattgttgttctcatggacattgttcacagccatgcatcaaataatactttagatggactgaacatgtttgacggcacagatagttgttactttcactctggagctcgtggttatcattggatgtgggattcccgcctctttaactatggaaactgggaggtacttaggtatcttctctcaaatgcgagatggtggttggatgagttcaaatttgatggatttagatttgatggtgtgacatcaatgatgtgtactcaccacggattatcggtgggattcactgggaactacgaggaatactttggactcgcaactgatgtggatgctgttgtgtatctgatgctggtcaacgatcttattcatgggcttttcccagatgcaattaccattggtgaagatgttagcggaatgccgacattttgtattcccgttcaagatgggggtgttggctttgactatcggctgcatatggcaattgctgataaatggattgagttgctcaagaaacgggatgaggattggagagtgagtgatattgttcatacactgacaaatagaagatggtcggaaaagtgtgtttcatacgctgaaagtcatgatcaagctctagtcggtgataaaactatagcattctggctgatggacaaggatatgtatgattttatggctctggatagaccgtcaacatcattaatagatcgtgggatagcattgcacaagatgattaggcttgtaactatgggattaggaggagaagggtacctaaatttcatgggaaatgaattcggccaccccgagtggattgatttccctagggctgaacaacacctctctgatggctcagtaattcccggaaaccaattcagttatgataaatgcagacggagatttgacctgggagatgcagattatttaagataccgtgggttgcaagaatttgaccgggctatgcagtatcttgaagataaatatgagtttatgacttcagaacaccagttcatatcacgaaaggatgaaggagataggatgattgtatttgaaaaaggaaacctagtttttgtctttaattttcactggacaaaaagctattcagactatcgcataggctgcctgaagcctggaaaatacaaggttgccttggactcggatgatccactttttggtggctttgggagaattgatcataatgccgaatacttcacctttgaaggatggtatgatgatcgtcctcgttcaattatggtgtatgcacctagtaggacagcagtggtctatgcactagtagacaaagaagcagaagaagaagtagcagtagtagaagaagaatgaacgaacttgtg

SEQ ID NO: 2 - DNA, 770 bp, Artificial Sequence
  Description of Artificial Sequence 5′ end SBEII cDNA
ttgatggggccttgaactcagcaatttgacactcagttagttacactcctatcactcatcagatctctatttttttctcttaattccaaccaaggaatgaattaaaagattagatttgaaggagagaagaagaaagatggtgtatacactctctggagtgcgttttcctactgtgtccatcagtgtacaatctaatggattcagcagtaatggtgatcggaggaatgctaatgtttctgtattcttgaaaaagcactctctttcacatcttggcgaaaagtcttcttacgattccgaatcccgaccttctacagttgcagcatcggggaaggtccttgtacctkgaahccmgagtgatagcycyycatyccycaacmgaccaatttgagttcactgagacagctccagaaattccccagcatcaactgatgtagatagttcaacaatggaacacgctagccagattaaaactgagaacgatgacgttgagccgtcaagtgatcttacaggaagtgttgaagagttggattttgcttcatcactacaactacaagaaggtggtaaactggaggagtctaaaacattaaatacttctgaagaracaattattgatgaatctgataggatcagagagaggggcatccctccaccytggacttggtcagaagatttatgaaatagacccccttttgacaaactatcgtcaacaccttgattacaggtattcacagtacaagaaaatgagggaggcaattgacaagtatgagggt

SEQ ID NO: 3 - DNA, 728 bp, Artificial Sequence
  Description of Artificial Sequence 5′ end of SBEII cDNA
ttgatggggccttgaactcagcaatttgacactcagttagttacactgccatcacttatcagatctctattttttctcttaattccaaccaaggaatgaataaaagatagatttgtaaaaccctaaggagagaagaagaaagatggtgtatacactctctggagtgcgttttcctactgttccatcagtgtacaaatctaatggattcagcagtaatggtgatcggaggaatgctaatatttctgtattcttgaaaaaacactctctttcacttgcagcatcggggaaagtccttgtgcctsgaacccagagtgatagctcctcatcctcaacagaccaatttgagttcactgagacatctccagaaaatcccccagcatcaactgatgtagatagttcaacaatggaacacgctagccagattaaaactgagaacgatgacgttgagccgtcaagtgatcttacaggaagtgttgaagagctggattttgcttcatcactacaactacaagaaggtggtaaactggaggagtctaaaacattaaatacttttgaagagacaattattgatgaatctgataggatcagagagaggggcatccctccacctggacttggtcagaagatttatgaaatagaccccccttttgacaaactatcgtcaacaccttgattacaggtattcacagtacaagaaactgagggaggcaattgacaagtatgagggt

SEQ ID NO: 4 - DNA, 352 bp, Artificial Sequence
  Description of Artificial Sequence SBE cDNA
ttgatggcaataatggagcatgcgtattatgccagttttggataccacgtgactaacttctttgccatcagctctcgctgtgggccacccgaggatctcaagtacctcatcgataaggcacatcagctcggattgtacgtccycatggatgtcgtccattcacatgcctccagtaactccatggacggtatcaataacttcgatggcacagaccaccagtacttccatgaaggagaacgtggaagacattcgttgtgggattctaggctcttcaactatggtcattgggaggttcttcgcttcctgctctccaatcttagatggtatatggaggagtatcactttgacggct

SEQ ID NO: 5 - DNA, 352 bp, Artificial Sequence
  Description of Artificial Sequence SBE cDNA
agccgtcaaagtgatactcctccatataccatctaagattggagagcaggaagcgaagaacctcccaatgaccatagttgaagagcctagaatcccacaacgaatgtcttccacgttctccttcatggaagtactggtggtctgtgccatcgaagttattgataccgtccatggagttactggaggcatgtgaatggacgacatccatgrggacgtacaatccgagctgatgtgccttatcgatgaggtacttgagatcctcgggtggcccacagcgagagctgatggcaaagaagttagtcacgtggtatccaaaactggcataatacgcatgctccattattgccatcaa

SEQ ID NO: 6 - DNA, 3753 bp, Artificial Sequence
  Description of Artificial Sequence SBE cDNA
gcccgkgcacccgkgccgaaaatcaaaccccgatgatgycsgcsccgggcgttgaataaacaaaagatacaaacattcacaatccgcatgcttttgtacataaacttactgtagtgcgccacgcggatgatgaccatgcattactcctgcatgatgtgatggcgccagttgcgcgggcgccctgccgtgacgctgcgcagtcgcgagatgtgcacgcgctggagcacgtgcgcctcgttaaattaaaaatgattcatgtttactatgcgaaaatttaaaaacaaatctgatacaacgattccgaagtgtctcttccgtggcgtcgctcaaagcgcgaccccaaaatagtgcacaatgcacamagatacatttttgtacatctccatatgtacaaagggcccaggcacaagcagcgtccgttgtgggcgcacgagcccaacagttgccagccgctcgccaagcgcctggaagcggagcacctccaagcctgcgccttttcaatgcacatgcctattccgttggcaacttgttgcacggccgtgtttctccgaagagcgctggcgctcatgtgcgcgtggccatgcgatcgactctcccgccgttgcccaaacaaccattctgcccttgtggccttaccttcacgctgcgcacttcaccagttgccattctctcagcgcgatgatttaaacaacgaatgacttcgctgttcacattgtgatcaaaaaacggtgggtaacacacgtgtgcctcgtggcgcgtccagatacgcggcgtgtgcacagccgcacgctgcacgcgcgtgccgccaccgctgtcgcgctcgctgtgtggctcagttcttcggatcgtgtcagccgtggcactgcccattcgccatttcactcgcacacagccgctcattgcagcccaactgcatgtcctgattcacttaacctccgctttatttgcgcaccccaaatccacccctcgcacacactcacttcctccacacctgcttcccgctcgcctaccgctcccccatcacttccccgtcaccatgggctcggaagacccccactactccgcctggaaggataagaaagacggcaccgcatgcatccgcgatgatcgctacttggagccctttgcagatgccctgcgttatcgctatgccaagttttccgagattctgtccgccatcgagtccagcgaaaagtccctcgacaacttttcacgcggatataaaactatgggaatccagcgcgtcaagggaggcattcgctaccgcgaatggggcacccagcgcccgcgaaaatgttcttctttggcgagtttaacaactgggatcgtcactctgtccctatgcagcgcgatgaatttggcatatggtcctgcttcattccgsgatcctgaaccgggcgtcccggctattaagcacggaaccaaggtgaaggctgccgttgtgccccatcacggtccgtggctcgaccgcaacccggcgtgggcaaccttctgcgtgcaagacccgcacacatttctgtatgataccgtcttctggaaccccccggagaagtacaagtggattggtccagatcacgtcaagcgaccctcttcgcttcgtatttacgagtgtcatgtcggcatgggcactaacgatctaaaggtcggctcatacagagagtttgctgacaacgttcttccgcgtattaagaacactggctacactgctttgcaaatcatggccattatggagcatgcgtattatgccagttttggataccacgtgactaacttctttgccaatmgctctcgctgtgggccacccgaggatctcaagtacctcatcgataaggcacatcagctcggattgtacgtcctcatggatgtcgtccattcacatgcctccagtmmctccatggacggtatcaataacttcgatggcacagaccaccagtacttccatgaaggagaacgtggaagacattcgttgtgggattctaggctcttcaactatggtcattgggaggttcttcgcttcctgctctccaatcttagatggtatatggaggagtatcactttgacggcttccgtttcgacggtatcacttccatgttgtaccttcattctggcattggtgtgcagttcactggcaactataatgaatactttggactgcaggttgatgttgatgcttgtgtttatatgatgcttgccaataagttacttcatgacttgcatcctgatgtggccgtgactatcgctgaagacgtcagcggcatgcctactctttgtgtccctgttgaacatggtggactcggtttcgactaccgtttggccatggccattcccgacatgtggatcgaggttcttgaaaaggagaaggacgagaattggaatatgggtaatattgtgttcactcttaccaatcgccgttggaacgagaagaccattgggtactgcgaaagtcatgatcaagcgttggttggggacaagactattgccttttggcttatggatgccgctatgtacactgatatgagctgcgatggatacccttcccccaccatcgagcgtggaattgcgctgcacaagttgatcagactgttgaccatgtgcttatcgggtgaaggttaccttacgttcatgggcaatgagtttggtcatccagaatggattgatttcccgcgtgaaggaaacggcaactcttaccaacatgcacgtcgtcgctgggatctttgcgacaatcagtcgctacgttataagcatttgtacgcttttgagaagatggcgcaccctttggacgacgaacatccgttcctccatttccaggcacatcagtatgtcgttttgtcacacgaaggtgacaagctggtcgtcgttgagaagggcgatcgtctgctgttcgtgtttaacttccatcataatcaatcgtattctgattatcgtattggcacttattggggtggtcggtataagttggttcttgattcggatggcacgaacactggtggacatggaagagtgcactgggatgtggtccatacgacgaatactgaaccgtggcataacagaccatactacatacaagtgtatgctcctgcacgcacgtgccaggtgtactattgctatgagacgtgggaggaagctgcgcagaaagaggggaaggcagccgacgcgcaaagcaagcctgcaactgtagtgactgagatagtggataagaaagcagcttctgagaaggctgtctctggcgctgaggatgttgagcaagtagcgtcgaaactacgcgcaaagctgacgacgaaagcagcggtcaaggctgttcagaaggaagctgtgtgagggacattgcttagatgtcggtggggtgttgagctcccatgaggagaacggggtatactctaacaaagtttgtaggctagatatgtgggctttctgagagccagtacgaactacaagcttcaagtgggtcccgttgatgcaggcgggtttgtttgaatgagcgttacagtgacaaaccgctgcacagatgaataccatggggtctgccgacggtaggttttttgatgggtatgatatcagttgttctttgttttggtctgttcgtcttaggctgaacgctgaaaagctttctgcgatgcctttgttgttgcgatgagtacgaacttgagtattttgtgcagttcggaaacttttgttgacagatgataaacaatactagtcttgtgatttgaatgaccataccctcaagttggggcgaattcgatatcaagctt

SEQ ID NO: 7 - DNA, 17 bp, Artificial Sequence
  Description of Artificial Sequence Nucleotides adjacent to ATG
aaggagatataacaatg

SEQ ID NO: 8 - DNA, 10 bp, Artificial Sequence
  Description of Artificial Sequence Nucleotides adjacent to ATG
gtcgaccatg

SEQ ID NO: 9 - DNA, 23 bp, Artificial Sequence
  Description of Artificial Seqeunce Oligonucleotide primer
tcagcagtaatggtgatcggagg

SEQ ID NO: 10 - DNA, 26 bp, Artificial Sequence
  Description of Artificial Sequence Oligonucleotide primer
cacaagttcgttcattcttcttctac

SEQ ID NO: 11 - DNA, 33 bp, Artificial Sequence
  Description of Artificial Sequence Oligonucleotide primer
cgggatcccgtcagcagtaatggtgatcggagg

SEQ ID NO: 12 - DNA, 29 bp, Artificial Sequence
  Description of Artificial Sequence Oligonucleotide primer
cgggatcccgaccgataatccgtggtgag

SEQ ID NO: 13 - DNA, 32 bp, Artificial Sequence
  Description of Artificial Sequence Oligonucleotide primer
cccaagcttcccgtctgtaagcatcattagtg

SEQ ID NO: 14 - DNA, 31 bp, Artificial Sequence
  Description of Artificial Sequence Oligonucleotide primer
ccaatgcatagggcgagggtatttgaagtgg

SEQ ID NO: 15 - DNA, 22 bp, Artificial Sequence
  Description of Artificial Sequence Oligonucleotide primer
ttgatggggccttgaactcagc

SEQ ID NO: 16 - DNA, 24 bp, Artificial Sequence
  Description of Artificial Sequence Oligonucleotide primer
accctcatacttgtcaattgcctc

SEQ ID NO: 17 - DNA, 35 bp, Artificial Sequence
  Description of Artificial Sequence Oligonucleotide primer
taggcgagctcaccctcatacttgtcaattgcctc

SEQ ID NO: 18 - DNA, 29 bp, Artificial Sequence
  Description of Artificial Sequence Oligonucleotide primer
ccaatgcatttgatggggccttgaactca

SEQ ID NO: 19 - DNA, 32 bp, Artificial Sequence; identical to SEQ ID NO: 13
  Description of Artificial Sequence Oligonucleotide primer

SEQ ID NO: 20 - DNA, 37 bp, Artificial Sequence
  Description of Artificial Sequence Oligonucleotide primer
ccaatgcatagcggaaatagctgaactgtgcttcatc

SEQ ID NO: 21 - DNA, 29 bp, Artificial Sequence
  Description of Artificial Sequence Oligonucleotide primer
ccaatgcatttgatggggccttgaactca

SEQ ID NO: 22 - DNA, 33 bp, Artificial Sequence
  Description of Artificial Sequence Oligonucleotide primer
taggcgagctcgccaagatgtgaaagagagtgc

SEQ ID NO: 23 - DNA, 30 bp, Artificial Sequence
  Description of Artificial Sequence Oligonucleotide primer
tctgatcatggcgatcatatcatggagaca

SEQ ID NO: 24 - DNA, 30 bp, Artificial Sequence
  Description of Artificial Sequence Oligonucleotide primer
ccgatcgaaagatccggaaagatcccgatc

SEQ ID NO: 25 - protein, 7 aa, Artificial Sequence
  Description of Artificial Sequence SBE motif
LMAIMEH

SEQ ID NO: 26 - protein, 8 aa, Artificial Sequence
  Description of Artificial Sequence SBE motif
FDGFRFDG

SEQ ID NO: 27 - DNA, 23 bp, Artificial Sequence
  Description of Artificial Sequence Oligonucleotide primer
ggcgcgccgggctcggaagaccc

SEQ ID NO: 28 - DNA, 26 bp, Artificial Sequence
  Description of Artificial Sequence Oligonucleotide primer
ggcgcgcctcacacagcttccttctg

SEQ ID NO: 29 - DNA, 30 bp, Artificial Sequence
  Description of Artificial Sequence Oligonucleotide primer
gaagatctatccgatcgtatcgatagagac

SEQ ID NO: 30 - DNA, 28 bp, Artificial Sequence
  Description of Artificial Sequence Oligonucleotide primer
gaagatctatcattctgcctcccgaacc